(19) United States
(12) Patent Application Publication
(10) Pub. No.: US 2010/0197776 A1
(43) Pub. Date: Aug. 5, 2010

(54) DIRECT DISSOLUTION OF DOCETAXEL IN A SOLVENT IN POLYSORBATE 80

(75) Inventors: Eric DIDIER, Paris (FR); Elie FOUQUE, Saint Maur des Fosses (FR); Mostafa NAKACH, Thiais (FR); Jean-René AUTHELIN, Saint Germain les Arpajon (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: AVENTIS PHARMA SA, Antony (FR)

(21) Appl. No.: 12/623,547

(22) Filed: Nov. 23, 2009

Related U.S. Application Data
(63) Continuation of application No. PCT/FR2008/000766, filed on Jun. 6, 2008.

(30) Foreign Application Priority Data
Jun. 8, 2007 (FR) 0704095

(51) Int. Cl. A61K 31/337; A61P 35/00

(52) U.S. Cl. 514/449

(57) ABSTRACT

The disclosure relates to the solubilization of docetaxel in an organic solvent, to the mixture thereof with polysorbate 80, and to the evaporation of the solvent.

This application is a continuation of International Application No. PCT/FR2008/000766, filed Jun. 6, 2008, which is incorporated herein by reference in its entirety; which claims the benefit of priority of French Patent Application No. 0704095, filed Jun. 8, 2007.

The present invention relates to a novel process for the preparation of a solution of docetaxel in polysorbate 80.

It relates, according to a first embodiment of the invention, more particularly to the solubilization of docetaxel in an organic solvent, to its mixing with polysorbate 80 and to the evaporation of the solvent.

The direct solubilization of docetaxel in polysorbate, even if it is possible, is a difficult stage. It requires extremely efficient stirring systems or an increase in the temperature which is harmful to the active principle. To date, solutions of docetaxel in polysorbate 80 have been prepared by a three-stage process, the first stage constituting the dissolution of the docetaxel in ethanol, followed by mixing with the polysorbate and then, finally, by the evaporation of the ethanol.

It is apparent that the ethanol used in the final formulation of the commercial composition comprising docetaxel was not the only solvent which can be used in the context of the present invention. Thus, numerous solvents capable of solubilizing docetaxel and which are miscible in all proportions with the polysorbate can be used. Use may thus be made of solvents exhibiting a boiling point of between 40 and 153° C. at atmospheric pressure; mention may be made, among these solvents, of chloroalkanes and in particular dichloromethane or chloroform, amides, such as dimethylformamide or dimethylacetamide, esters, such as ethyl acetate, ketones, such as acetone or methyl isobutyl ketone, or nitriles, such as acetonitrile. The preferred solvents are chosen from acetone, acetonitrile, methylene chloride or dimethylformamide. The docetaxel used as starting material in the context of the present invention can be an amorphous docetaxel or a docetaxel crystallized in any form, such as an acetonate, an alcoholate, a hydrate or a crystal with acetonitrile.

The process according to the invention is not limited to the dissolution of docetaxel in the solid form in a solvent, followed by the addition of polysorbate and distillation of the solvent, but can also consist in using the docetaxel solution obtained at the outlet of a purification column. This solution can be a solution of docetaxel in a single solvent, such as ethyl acetate, acetone, methylene chloride or tetrahydrofuran, but can also be a solution in a mixture of the abovementioned solvents. This column is generally composed of a column of silica but any other material which makes purification possible can be used. We prefer, in the context of the present invention, to use a silica and in particular a silica sold under the Lichrospher trademark. Entirely preferably, use is made of a Lichrospher silica exhibiting a particle diameter of 12 μm.

The docetaxel solution to be purified is preferably a solution of docetaxel in ethyl acetate or in a mixture of ethyl acetate with a hydrocarbon, such as cyclohexane, hexanes or toluene. The solution resulting from the purification column, if the docetaxel present has the required purity, can be mixed directly with the polysorbate and then the solvent(s) can be evaporated without an intermediate stage of crystallization of docetaxel in any solvate form. This exhibits a considerable advantage from an economic viewpoint.

The present invention will be more fully described with the help of the following examples, which should not be regarded as limiting the invention.

### EXAMPLE 1 (FTA 152)

4.3320 g of docetaxel trihydrate are dissolved in 37.9 g of absolute ethanol, 108.0 g of polysorbate 80 are added dropwise and a considerable foam appears. Distillation is carried out under a pressure of 50 mbar with a bath temperature of 40° C. After distilling for 4 hours 10 minutes, the following are obtained: 33.9 g of distillate and 167.8 g (to be confirmed) of solution of docetaxel in the polysorbate comprising less than 0.01% of ethanol and 0.28% of impurities.

### EXAMPLE 2 (FTA 153)

4.2017 g of docetaxel in the acetonitrile solvate form are dissolved in 533 ml of acetonitrile (419.2 g), and 108.0 ml of polysorbate 80 are added dropwise. Dissolution is carried out under a mean pressure of 55 mPa with a bath temperature of 40° C. After distilling for 6 hours 25 minutes, the following are obtained: 99.2 g of solution of docetaxel in the polysorbate comprising 0.06% of acetonitrile and 0.41% of impurities.

The acetonitrile solvate is prepared in the following way:

The process consists of the deprotection (detrocing) of docetaxel diprotected in the 7 and 10 positions to give docetaxel, which is isolated by crystallization from a toluene/acetonitrile mixture.

The following are charged to a 1 l reactor: 900 ml of ethyl acetate, 7.8 mg of 4-methoxyphenol and 78 g of docetaxel diprotected by a trichloroethoxycarbonyl group. The reaction medium is stirred and then 120 ml of ethyl acetate are distilled off under reduced pressure. Back at 23° C., 37 g of zinc are charged. 74 g of acetic acid are then run in while maintaining the temperature at 25±2° C. It takes 1 h 15 to run in the acetic acid. Stirring is maintained for 1 h 15, at the end of which time the reaction is complete.

The reaction medium is filtered under nitrogen (zinc cake) and the cake is washed three times with ethyl acetate. The mother liquors and the wash liquors are combined and then they are washed with water and then with an aqueous sodium bicarbonate solution. A solution of 7.2 mg of 4-methoxyphenol in 2 ml of ethyl acetate is charged to the organic phase and then washing is carried out with water.

Subsequently, a change in solvent to acetonitrile is carried out. At the end of the change in solvent, the temperature is brought back to 25° C. and then 113 ml of toluene are run in over 2 h. Stirring is maintained at this temperature overnight and then the reaction medium is cooled to 0° C. over 3 h. The slurry obtained is filtered at 0° C. The cake is rinsed with cold toluene.

The cake thus obtained is dried in an oven to constant weight (27 h).

51.7 g of a white powder are thus obtained.

| Determinations | Test |
|---|---|
| RY assayed % | 87.8 |
| Water % | 0.6 |
| Acetonitrile | 4.7 |
| Ethyl acetate | 0.2 |
| Sum of the solvents | 5.5 |
| Content with regard to as is % | 95.2 |
| Content on a dry basis % | 100.8 |
| Sum of the impurities % | 0.73 |

### EXAMPLE 3 (FTA 154)

4.3324 g of docetaxel in the trihydrate form are dissolved in 85 g of dimethylformamide, and 108.0 g of polysorbate 80 are added dropwise. Distillation is carried out under a pressure of 48 mPa with a bath temperature of 67° C. After distilling for 6 hours 15 minutes, the following are obtained: 106.1 g (to be confirmed) of solution of docetaxel in the polysorbate comprising 0.01% of dimethylformamide and 0.43% of impurities.

### EXAMPLE 4 (FTA 155)

4.1792 g of docetaxel in the acetonate form are dissolved in 21 ml of acetone, and 108.0 g of polysorbate 80 are added dropwise. Distillation is carried out under a pressure of 46 mbar with a bath temperature of 38° C. After distilling for 4 hours 45 minutes, the following are obtained: 99.7 g of solution of docetaxel in the polysorbate comprising 0.01% of acetone and 0.34% of impurities.

### EXAMPLE 5 (FTA 156)

4.33 g of docetaxel trihydrate are dissolved in 165.4 g of dichloromethane, and 108.0 g of polysorbate 80 are added dropwise. Distillation is carried out under a pressure of 84 mbar with a bath temperature of 38° C. After distilling for 5 hours 5 minutes, the following are obtained: 101.2 g of solution of docetaxel in the polysorbate comprising 0.11% of dichloromethane and 0.35% of impurities.

### EXAMPLE 6 (FTA 151)

148.1 g of docetaxel resulting from the purification on a silica column and dissolved in ethyl acetate at a concentration of 2.7% by weight/weight are mixed, and 108.2 g of polysorbate 80 are added dropwise. Distillation is carried out under a pressure of 55 mbar with a bath temperature of 40° C. After distilling for 3 hours 15 minutes, the following are obtained: 95.1 g of solution of docetaxel in the polysorbate comprising 0.01% of dichloromethane by weight and 0.64% of impurities.

What is claimed is:

1. A process for preparing a solution of docetaxel in polysorbate 80, comprising dissolving docetaxel in an organic solvent having a boiling point of between 40 and 153° C., with the exception of ethanol; mixing the solution obtained with polysorbate 80; and evaporating the organic solvent under reduced pressure.

2. The process according to claim 1, wherein the docetaxel is in crystalline form.

3. The process according to claim 2, wherein the docetaxel is in the trihydrate form, acetonate form or acetonitrile solvate form.

4. The process according to claim 1, wherein the organic solvent is chosen from the group consisting of acetone, acetonitrile, methylene chloride and DMF.
